SEQUENCE LISTING (15 sequences)

SEQ ID NO: 1 - DNA, 40 bp, Artificial Sequence
  Synthetic Construct
atgccgaagcccgccgctgggttttagagctagaaatagc

SEQ ID NO: 2 - DNA, 40 bp, Artificial Sequence
  Synthetic Construct
ccagcggcgggcttcggcatactagtattatacctaggac

SEQ ID NO: 3 - DNA, 2117 bp, Artificial Sequence
  Synthetic Construct
catgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagtatgccgaagcccgccgctgggttttagagctagaaatagcaagttaaaataaggctagtccgttatcaacttgaaaaagtggcaccgagtcggtgctttttttgaattctctagagtcgacctgcagaagcttagatctattaccctgttatccctactcgagttcatgtgcagctccataagcaaaaggggatgataagtttatcaccaccgactatttgcaacagtgccgttgatcgtgctatgatcgactgatgtcatcagcggtggagtgcaatgtcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataagatgccgctcgccagtcgattggctgagctcataagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 4 - DNA, 16 bp, Artificial Sequence
  Synthetic Construct
ccaggcagagcaagtt

SEQ ID NO: 5 - DNA, 31 bp, Artificial Sequence
  Synthetic Construct
gggaagttgtaagtcatctcacgcatttcag

SEQ ID NO: 6 - DNA, 33 bp, Artificial Sequence
  Synthetic Construct
aaatgcgtgagatgacttacaacttcccacagg

SEQ ID NO: 7 - DNA, 19 bp, Artificial Sequence
  Synthetic Construct
gctccaccagacactccgt

SEQ ID NO: 8 - DNA, 17 bp, Artificial Sequence
  Synthetic Construct
gtgattgccctgagtga

SEQ ID NO: 9 - DNA, 17 bp, Artificial Sequence
  Synthetic Construct
tgaagatgtgggtcctg

SEQ ID NO: 10 - DNA, 36 bp, Artificial Sequence
  Synthetic Construct
ctaaggaggttataaaaaatgaagccttctatcgtt

SEQ ID NO: 11 - DNA, 35 bp, Artificial Sequence
  Synthetic Construct
aaggcgaagcggcatgctttcagcatcacgccgcg

SEQ ID NO: 12 - DNA, 36 bp, Artificial Sequence
  Synthetic Construct
acgtggctttccctgcagtaggggttccgcgcacat

SEQ ID NO: 13 - DNA, 36 bp, Artificial Sequence
  Synthetic Construct
aacgatagaaggcttcattttttataacctccttag

SEQ ID NO: 14 - DNA, 35 bp, Artificial Sequence
  Synthetic Construct
cgcggcgtgatgctgaaagcatgccgcttcgcctt

SEQ ID NO: 15 - DNA, 36 bp, Artificial Sequence
  Synthetic Construct
atgtgcgcggaacccctactgcagggaaagccacgt

(19) United States
(12) Patent Application Publication
(10) Pub. No.: US 2025/0019711 A1
(43) Pub. Date: Jan. 16, 2025

(54) METHOD FOR CONSTRUCTING STRAIN FOR PRODUCING RECOMBINANT PROTEIN CONTAINING UNNATURAL AMINO ACIDS, AND STRAIN OBTAINED THEREFROM

(71) Applicant: Novocodex Biopharmaceuticals Co., Ltd., Yuechen District Shaoxing, Zhejiang (CN)

(72) Inventors: Longfei Chen, Yuechen District Shaoxing, Zhejiang (CN); Lin Jiao, Yuechen District Shaoxing, Zhejiang (CN); Yuebin Ying, Yuechen District Shaoxing, Zhejiang (CN); Zunyang Gong, Yuechen District Shaoxing, Zhejiang (CN); Jingjing Zhu, Yuechen District Shaoxing, Zhejiang (CN); Xuejun Liang, Yuechen District Shaoxing, Zhejiang (CN)

(73) Assignee: Novocodex Biopharmaceuticals Co., Ltd., Yuechen District Shaoxing, Zhejiang (CN)

(21) Appl. No.: 18/293,728

(22) PCT Filed: Mar. 2, 2022
(86) PCT No.: PCT/CN2022/078826

§ 371 Date: Sep. 9, 2024

(30) Foreign Application Priority Data
Aug. 11, 2021 (CN) 202110917380.5

(51) Int. Cl. C12N 15/70; C07K 14/245; C07K 14/61; C12N 9/00; C12P 21/00

(52) U.S. Cl. CPC C12N 15/70; C07K 14/245; C07K 14/61; C12N 9/93; C12P 21/00; C12Y 601/01

(57) ABSTRACT

Provided in the present invention are a method for constructing a strain for producing a recombinant protein containing unnatural amino acids, and the strain obtained therefrom. The method comprises modifying the expression of a prfA gene contained in the strain to be controllable. The strain constructed by the method of the present invention can efficiently produce in intact protein containing unnatural amino acids, greatly reduce the production of a truncated protein and can also maintain high-speed growth.

### CROSS-REFERENCE TO RELATED APPLICATIONS

This application is the 371 National Phase of International Application No. PCT/CN2022/078826, filed Mar. 2, 2022, which claims priority to and the benefit of the earlier filing of CN 202110917380.5, filed Aug. 11, 2021; each of which is incorporated by reference herein in its entirety.

### INCORPORATION BY REFERENCE OF SEQUENCE LISTING

A computer readable text file, entitled “S481-6000US_NewSeqList_ST25” created on or about Feb. 12, 2024, with a file size of 6,066 bytes, contains the sequence listing for this application and is hereby incorporated by reference in its entirety.

### TECHNICAL FIELD

The present invention belongs to the technical field of bioengineering, and relates to the construction of a strain for producing a recombinant protein containing unnatural amino acids.

### BACKGROUND

Amber codon (amber) UAG is a termination codon that is the least frequently used in Escherichia coli, accounting for approximately 7%. When this codon is modified to encode an unnatural amino acid, it has the least endogenous protein affected and is therefore the codon most commonly used to establish the gene codon expansion technology. A recombinant protein with site-directed insertion of unnatural amino acids can be produced by expressing an unnatural amino acid tRNA/tRNA synthetase in a normal Escherichia coli strain via a helper plasmid, and introducing the amber codon into a reading frame of the recombinant protein via an expression vector in a site-directed way. However, since a peptide chain releasing factor RF1 recognizing the amber codon as a termination codon exists in Escherichia coli, the yield of the recombinant protein with site-directed insertion of unnatural amino acids is low, and a large amount of truncated protein may be generated due to premature translation termination. The structure of some special recombinant proteins may even seriously hinder the insertion of unnatural amino acids at certain sites, and thus it is difficult to obtain an intact recombinant protein containing unnatural amino acids. Direct gene knockout of the encoding gene prfA of RF1 to inhibit the competition between RF1 and unnatural amino acids can cause a great reduction in the growth rate of the strain, and is hard to be applied to industrial fermentation production.

### SUMMARY

The present invention solves the above realistic problems by modifying the expression of RF1 protein in Escherichia coli.

In a first aspect, the present invention provides a method for constructing a strain for producing a recombinant protein containing unnatural amino acids, comprising: modifying the expression of a prfA gene contained in the strain to be controllable. To implement the method, the expression of the prfA gene in the genome of the strain may be modified to be either suppressed or inducible.

In one embodiment, the expression of the prfA gene in the genome of the strain is modified to be suppressed, and a plasmid containing the prfA gene and capable of expressing RF1 is introduced into the strain, wherein the expression or replication of the prfA gene in the plasmid is controlled.

Preferably, the promoter of the prfA gene in the plasmid is an inducible expression promoter, so as to achieve inducible expression. The inducible expression promoter may be, e.g., lactose-induced tac promoter (GenBank: K01728.1), lacUV5 promoter (GenBank: E02301.1), T7 promoter (GenBank: M38302.1), T5 promoter (GenBank: X00126.1), arabinose-induced araBAD promoter (GenBank: K00953.1), or thermo-induced pR/pL promoter (Winstanley, C., et al., “Differential regulation of lambda pL and pR promoters by a cI repressor in a broad-host-range thermoregulated plasmid marker system.” Applied and Environmental Microbiology, 55.4 (1989): 771-777), etc.

Preferably, the replication origin of the prfA gene in the plasmid is a conditional deletion-type replication origin, such that the plasmid stops replicating in specific conditions, thus achieving the purpose of controlling the expression of the prfA gene. The conditional deletion-type replication origin may be, e.g., high-temperature conditional deletion-type replication origin pSG5 (GenBank: NC_008792.1), pSC101 (GenBank: K00042.1), etc.

Preferably, the modification of the expression of the prfA gene in the genome of the strain to be suppressed is achieved by gene knockout, gene silencing, gene mutation, or the like, such that the prfA gene in the genome does not express RF1 protein. For example, gene knockout can be achieved by introducing loxP loci at both ends of the prfA gene and then using Cre recombinase at an appropriate time point to induce gene knockout.

In one embodiment, the expression of the prfA gene in the genome of the strain is modified to be inducible by replacing the promoter of the prfA gene in the genome by an inducible expression promoter.

Preferably, the strain is Escherichia coli.

Elements such as the gene sequence of the prfA gene (GenBank Gene ID: 949002), inducible expression promoters (e.g., lactose-induced tac promoter, lacUV5 promoter, T7 promoter, T5 promoter, arabinose-induced araBAD promoter, thermo-induced pR/pL promoter, etc.), and replication origin can be found by those skilled in the art in various gene databases.

More preferably, the method further comprises: introducing a plasmid encoding an unnatural amino acid tRNA/tRNA synthetase orthogonal to a natural amino acid tRNA/tRNA synthetase into the strain. As such, since amber codon (UAG) encodes unnatural amino acids, a recombinant protein with site-directed insertion of unnatural amino acids can be produced by site-directed insertion of the amber codon into a coding frame of the recombinant protein. The plasmid encoding an unnatural amino acid tRNA/tRNA synthetase orthogonal to a natural amino acid tRNA/tRNA synthetase may be, e.g., a pUltra-py1RS helper plasmid, as those skilled in the art may find the relevant information by using the addgene website ([[https://www.]]addgene.org/collections/genetic-code-expansion/).

In one preferred embodiment, provided is a method for constructing a strain for producing a recombinant protein containing unnatural amino acids, wherein the strain is Escherichia coli BL21 (DE3), and the method comprises:
(1) knocking out the prfA gene from the genome of the strain by CRISPR technology;
(2) constructing a plasmid containing a pSG5 replication origin, an araBAD promoter, and a prfA gene; and
(3) transferring the plasmid in step (2) and a helper plasmid expressing an unnatural amino acid tRNA/tRNA synthetase, such as pUltra-py1RS helper plasmid, into the strain with the prfA gene in the genome of the strain knocked out obtained in step (1).

In a second aspect, provided is a strain for producing a recombinant protein containing unnatural amino acids, wherein the strain is capable of expressing an unnatural amino acid tRNA/tRNA synthetase orthogonal to a natural amino acid tRNA/tRNA synthetase, and the expression of a prfA gene in the strain is controlled. Preferably, the strain is constructed by the method of the present invention.

In a third aspect, provided is a method for producing a recombinant protein containing unnatural amino acids, comprising: introducing a gene encoding the recombinant protein into the strain according to the present invention, and culturing the strain to express the recombinant protein.

Preferably, the method comprises: 1) constructing a strain with controlled prfA gene expression; 2) introducing a helper plasmid expressing an unnatural amino acid tRNA/tRNA synthetase and an expression plasmid expressing the target recombinant protein into the strain; 3) inducing the expression of the prfA gene during the strain growth stage; and inducing the prfA gene to stop expression and inducing the recombinant protein to express in the recombinant protein expression stage.

In the present invention, the expression of the encoding gene prfA of RF1 is controlled, and a strain nearly completely using UAG for encoding unnatural amino acids is obtained. During the strain expansion stage, the strain is controlled to produce sufficient RF1 to maintain the rapid growth of the strain, while during the expression induction stage, the prfA gene is not expressed, resulting in nearly 100% production capacity for producing an intact recombinant protein. The strain constructed by the method of the present invention can efficiently produce an intact recombinant protein containing unnatural amino acids, greatly reduce the production of a truncated protein and can also maintain a high-speed growth.

### BRIEF DESCRIPTION OF THE DRAWINGS

FIGS. 1A-1B shows the knockout of the prfA gene in Escherichia coli, wherein FIG. 1A is a schematic diagram and FIG. 1B is the results of gel electrophoresis in screening;

FIG. 2 shows a plasmid map of pRF1;

FIG. 3 shows the growth curves of three strains, NCB21 (DE3), BL21 (DE3) co-transformed with plasmid pRF1 and helper plasmid pUltra-py1RS and NCB21 (DE3)-pRF1-free;

FIG. 4 shows a comparison of the expression of proteins with site-directed insertion of unnatural amino acids.

### DETAILED DESCRIPTION

The present invention will be further illustrated with reference to the following specific examples. The experimental methods used in the following examples are conventional standard experimental methods unless otherwise stated. The experimental materials, reagents and the like used in the following examples are commercially available unless otherwise stated. It should be understood that these examples are merely intended to illustrate the present invention rather than limit the scope of the present invention. Experimental procedures without specified conditions in the following examples, are generally carried out in conventional conditions, for example, as described in Molecular Cloning: A Laboratory Manual, edited by Joseph Sambrook, et al., or in conditions recommended by the manufacturer.

The following examples provided an Escherichia coli strain that can efficiently produce a recombinant protein with site-directed insertion of unnatural amino acids by multi-step modification of the strain. In brief, the method comprises the following steps: (1) knocking out the prfA gene from the genome of the strain by CRISPR technology to give a strain using 100% amber codon UAG for encoding unnatural amino acids; (2) constructing a thermo-sensitive plasmid (containing a pSG5 replication origin), wherein the plasmid contains the prfA gene (containing an araBAD promoter), of which the expression is induced by arabinose, the plasmid is compatible with a helper plasmid expressing an unnatural amino acid tRNA/tRNA synthetase and an expression plasmid expressing a target recombinant protein but has a different selection marker; (3) co-transforming the strain obtained in step (1) with the plasmid constructed in step (2), the helper plasmid, and the recombinant protein expression plasmid to give the final strain; (4) adding arabinose into the culture medium to promote the growth of the strain, raising the culture temperature to stop the replication of the thermo-sensitive plasmid when entering the expression induction stage, and adding IPTG to induce the expression of the recombinant protein and the site-directed insertion of unnatural amino acids to produce the recombinant protein containing unnatural amino acids.

### Example 1. Construction of prfA Gene-Knockout Plasmid

The prfA gene on the genome (GenBank: AM946981.1) of BL21 (DE3) strain (Thermo Scientific™, Cat #EC0114) was knocked out using CRISPR technology. The specific procedures were as follows:

First, a DNA fragment of about 2.5 kb in length was amplified using two primers, i.e., primer prfA-tgF (SEQ ID NO. 1) and primer prfA-tgR (SEQ ID NO. 2), using plasmid pTargetF (ADDGENE #62226) as the PCR template. After purifying the PCR product, the template DNA was digested with DpnI restriction endonuclease, the digested product was purified and collected, and then transformed into TOP10 competent cells. The recombinant transformants were screened using spectinomycin. The plasmid pTarget-prfA was obtained by selecting the recombinant transformants, extracting the plasmid, and sequencing. The sequence is set forth in SEQ ID NO. 3. The guide gRNA expressed by the plasmid can guide Cas9 protein to cut off double-stranded DNA at the target position of the prfA gene on the genome.

| | |
|---|---|
| prfA-tgF | ATGCCGAAGCCCGCCGCTGGGTTTTAGAGCTAGAAATAGC (SEQ ID NO. 1) |
| prfA-tgR | CCAGCGGCGGGCTTCGGCATACTAGTATTATACCTAGGAC (SEQ ID NO. 2) |

SEQ ID NO. 3
Plasmid pTarget-prfA
CATGTTCTTTCCTGCGTTATCCCCTGATTCTGTGGATAACCGTATTACCGC
CTTTGAGTGAGCTGATACCGCTCGCCGCAGCCGAACGACCGAGCGCAG
CGAGTCAGTGAGCGAGGAAGCGGAAGAGCGCCTGATGCGGTATTTTCTC
CTTACGCATCTGTGCGGTATTTCACACCGCATATGCTGGATCCTTGACAG
CTAGCTCAGTCCTAGGTATAATACTAGTATGCCGAAGCCCGCCGCTGGGT
TTTAGAGCTAGAAATAGCAAGTTAAAATAAGGCTAGTCCGTTATCAACTT
GAAAAAGTGGCACCGAGTCGGTGCTTTTTTTGAATTCTCTAGAGTCGAC
CTGCAGAAGCTTAGATCTATTACCCTGTTATCCCTACTCGAGTTCATGTGC
AGCTCCATAAGCAAAAGGGGATGATAAGTTTATCACCACCGACTATTTGC
AACAGTGCCGTTGATCGTGCTATGATCGACTGATGTCATCAGCGGTGGA
GTGCAATGTCATGAGGGAAGCGGTGATCGCCGAAGTATCGACTCAACTA
TCAGAGGTAGTTGGCGTCATCGAGCGCCATCTCGAACCGACGTTGCTGG
CCGTACATTTGTACGGCTCCGCAGTGGATGGCGGCCTGAAGCCACACAG
TGATATTGATTTGCTGGTTACGGTGACCGTAAGGCTTGATGAAACAACGC
GGCGAGCTTTGATCAACGACCTTTTGGAAACTTCGGCTTCCCCTGGAGA
GAGCGAGATTCTCCGCGCTGTAGAAGTCACCATTGTTGTGCACGACGAC
ATCATTCCGTGGCGTTATCCAGCTAAGCGCGAACTGCAATTTGGAGAATG
GCAGCGCAATGACATTCTTGCAGGTATCTTCGAGCCAGCCACGATCGAC
ATTGATCTGGCTATCTTGCTGACAAAAGCAAGAGAACATAGCGTTGCCTT
GGTAGGTCCAGCGGCGGAGGAACTCTTTGATCCGGTTCCTGAACAGGAT
CTATTTGAGGCGCTAAATGAAACCTTAACGCTATGGAACTCGCCGCCCG
ACTGGGCTGGCGATGAGCGAAATGTAGTGCTTACGTTGTCCCGCATTTG
GTACAGCGCAGTAACCGGCAAAATCGCGCCGAAGGATGTCGCTGCCGA
CTGGGCAATGGAGCGCCTGCCGGCCCAGTATCAGCCCGTCATACTTGAA
GCTAGACAGGCTTATCTTGGACAAGAAGAAGATCGCTTGGCCTCGCGCG
CAGATCAGTTGGAAGAATTTGTCCACTACGTGAAAGGCGAGATCACCAA
GGTAGTCGGCAAATAAGATGCCGCTCGCCAGTCGATTGGCTGAGCTCAT
AAGTTCCTATTCCGAAGTTCCGCGAACGCGTAAAGGATCTAGGTGAAGA
TCCTTTTTGATAATCTCATGACCAAAATCCCTTAACGTGAGTTTTCGTTCC
ACTGAGCGTCAGACCCCGTAGAAAAGATCAAAGGATCTTCTTGAGATCC
TTTTTTTCTGCGCGTAATCTGCTGCTTGCAAACAAAAAAACCACCGCTAC
CAGCGGTGGTTTGTTTGCCGGATCAAGAGCTACCAACTCTTTTTCCGAA
GGTAACTGGCTTCAGCAGAGCGCAGATACCAAATACTGTCCTTCTAGTG
TAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGT
CGTGTCTTACCGGGTTGGACTCAAGACGATAGTTACCGGATAAGGCGCA
GCGGTCGGGCTGAACGGGGGGTTCGTGCACACAGCCCAGCTTGGAGCG
AACGACCTACACCGAACTGAGATACCTACAGCGTGAGCTATGAGAAAGC
GCCACGCTTCCCGAAGGGAGAAAGGCGGACAGGTATCCGGTAAGCGGC
AGGGTCGGAACAGGAGAGCGCACGAGGGAGCTTCCAGGGGGAAACGC
CTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCA.

### Example 2. Construction of Homologous Recombinant DNA Fragments for Traceless Knockout of prfA Gene

Using the genomic DNA of BL21 (DE3) as the PCR template, a 5′ homology arm DNA fragment for homologous recombination during the knockout of the prfA gene was amplified using primer prfA-5F and primer prfA-5R. Using the genomic DNA of BL21 (DE3) as the PCR template, a 3′ homology arm DNA fragment for homologous recombination during the knockout of the prfA gene was amplified using primer prfA-3F and primer prfA-3R. The above two homology arm DNA fragments were purified and collected, mixed as an overlapping PCR template, and subjected to overlapping PCR using primer prfA-5F and primer prfA-3R to give a DNA fragment for homologous recombination, which contained about 0.48 kb of 5′ homology arm and about 0.45 kb of 3′ homology arm.

| | |
|---|---|
| prfA-5F | CCAGGCAGAGCAAGTT (SEQ ID NO. 4) |
| prfA-5R | GGGAAGTTGTAAGTCATCTCACGCATTTCAG (SEQ ID NO. 5) |
| prfA-3F | AAATGCGTGAGATGACTTACAACTTCCCACAGG (SEQ ID NO. 6) |
| prfA-3R | GCTCCACCAGACACTCCGT (SEQ ID NO. 7) |

### Example 3. Construction of prfA Gene-Deleted Strain

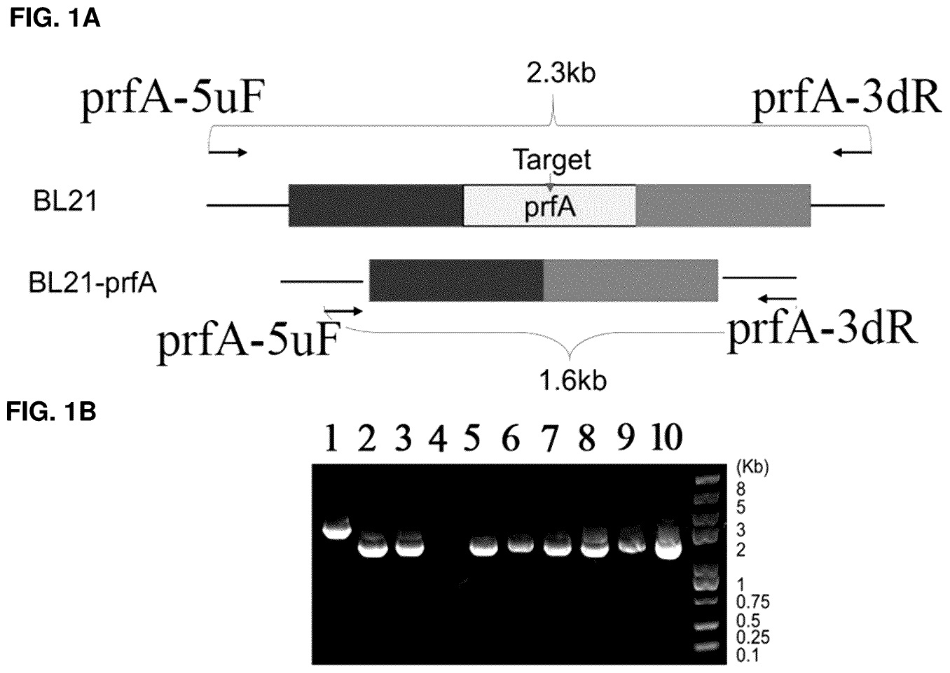

Plasmid pCAS (ADDGENE, #60847) was first introduced by transformation into BL21 (DE3) strain, and the resulting strain was used to prepare competent cells, and the medium was supplemented with 20 mM arabinose during the preparation of the competent cells to induce expression of the λ red system on the pCAS plasmid. The homologous recombination fragment and plasmid pTarget-prfA prepared above were used for co-transformation, and candidate single colonies were obtained by co-screening using kanamycin and spectinomycin. The candidate single colonies were subjected to colony PCR verification using primer prfA-5uF and primer prfA-3dR. As shown in FIG. 1A, the correct knockout product was about 1.6 kb, while the PCR product of strains with failed knockout was about 2.3 kb, and as shown in FIG. 1B, 8 out of 10 selected candidate single colonies exhibited correct knockout results. The strain with the prfA gene correctly knocked out was cultured. At 30° C., IPTG was added to the culture medium to induce pTarget-prfA plasmid elimination. Then, the pCAS plasmid was eliminated by increasing the culture temperature to 37° C. to finally give a prfA gene-deleted strain RI 21 (DF3)::ΔprfA without any plasmid

| | |
|---|---|
| prfA-5uF | GTGATTGCCCTGAGTGA (SEQ ID NO. 8) |
| prfA-3dR | TGAAGATGTGGGTCCTG (SEQ ID NO. 9) |

### Example 4. Construction of Plasmid with Inducible Expression of RF1 and Conditional Deletion

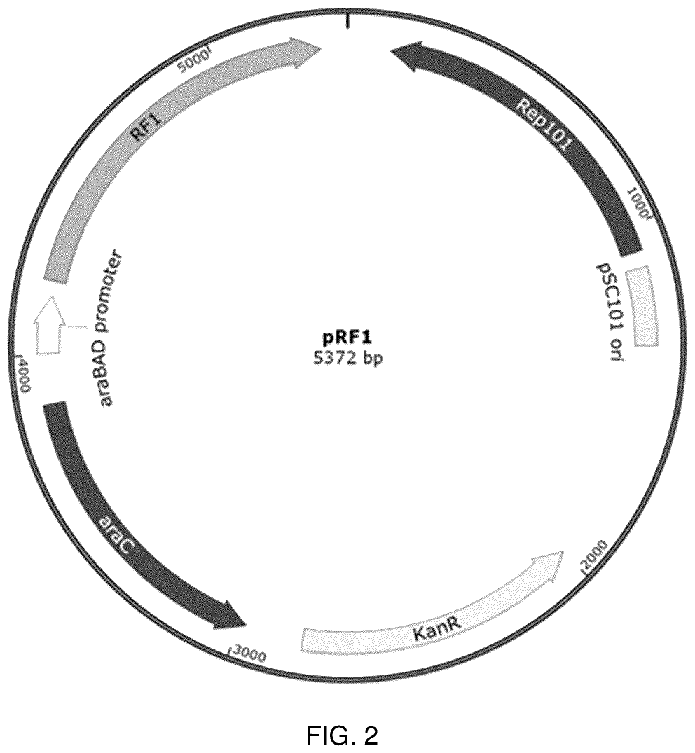

Using BL21 genomic DNA as the PCR template, the open reading frame and 3′ flanking sequence of the prfA gene were amplified using primers RF1-F and RF1-R to give a DNA fragment of about 1.15 kb. Using pCAS plasmid DNA as the PCR template, a DNA fragment of about 1.28 kb containing an araC gene and an araB promoter was amplified using primers Ara-F and Ara-R. Using pCAS plasmid DNA as the PCR template, a DNA fragment of about 2.93 kb containing a thermo-sensitive replication origin pSG5 and a kanamycin resistance gene was amplified using primers Rep101-F and Rep101-R. DNA fragments obtained by the 3 PCR amplifications were purified and collected, subjected to recombinant linkage with NEBuilder® HiFi DNA Assembly Master Mix reagents (NEB #E5520S), and TOP10 competent cells were transformed with the recombination product. The recombinants were screened using kanamycin and cultured at 30° C. Plasmids were extracted from the selected recombinants and subjected to enzyme digestion and sequencing verification to give the plasmid pRF1. The plasmid map is shown in FIG. 2.

| | |
|---|---|
| RF1-F | CTAAGGAGGTTATAAAAAATGAAGCCTTCTATCGTT (SEQ ID NO. 10) |
| RF1-R | AAGGCGAAGCGGCATGCTTTCAGCATCACGCCGCG (SEQ ID NO. 11) |
| Ara-F | ACGTGGCTTTCCCTGCAGTAGGGGTTCCGCGCACAT (SEQ ID NO. 12) |
| Ara-R | AACGATAGAAGGCTTCATTTTTTATAACCTCCTTAG (SEQ ID NO. 13) |
| Rep101-F | CGCGGCGTGATGCTGAAAGCATGCCGCTTCGCCTT (SEQ ID NO. 14) |
| Rep101-R | ATGTGCGCGGAACCCCTACTGCAGGGAAAGCCACGT (SEQ ID NO. 15) |

### Example 5. Acquisition of Escherichia coli Strain that can be Applied in Efficient Production of Recombinant Protein with Site-Directed Insertion of Unnatural Amino Acids

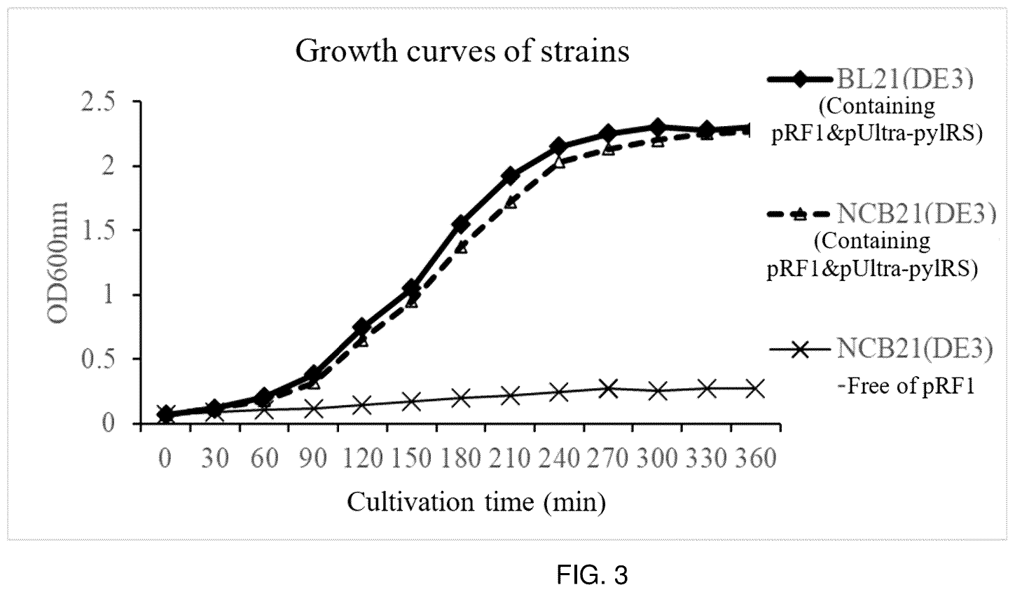

The BL21 (DE3)::ΔprfA competent cells obtained in Example 3 were co-transformed with the plasmid pRF1 and the helper plasmid pUltra-py1RS (Chatterjee A., Sun S. B., Furman J. L., et al., A Versatile Platform for Single- and Multiple-Unnatural Amino Acid Mutagenesis in Escherichia coli, [J]. Biochemistry, 2013, 52 (10): 1828-1837.). Transformants were co-screened using kanamycin and spectinomycin, and a strain with resistance to the two antibiotics was selected and designated as NCB21 (DE3). This strain can be used for efficient production of recombinant protein with site-directed insertion of unnatural amino acids. The strain was cultured and competent cells were prepared. The culture was performed at 30° C. using an LB culture medium supplemented with 1 mg/mL arabinose. The BL21 (DE3) strain co-transformed with plasmid pRF1 and helper plasmid pUltra-py1RS, NCB21 (DE3) strain (co-transformed with plasmids pRF1 and pUltra-py1RS), and the strain NCB21 (DE3)-pRF1-free with the prfA gene knocked out and only transformed with the helper plasmid pUltra-py1RS were compared, and the results are shown in FIG. 3. The results show mild difference in the growth between BL21 (DE3) and NCB21 (DE3), demonstrating that the pRF1 plasmid has a significant promoting effect on the growth of the prfA gene-deleted strain.

### Example 6. Use of NCB21 (DE3) Strain for the Production of Recombinant Protein with Site-Directed Insertion of Unnatural Amino Acids

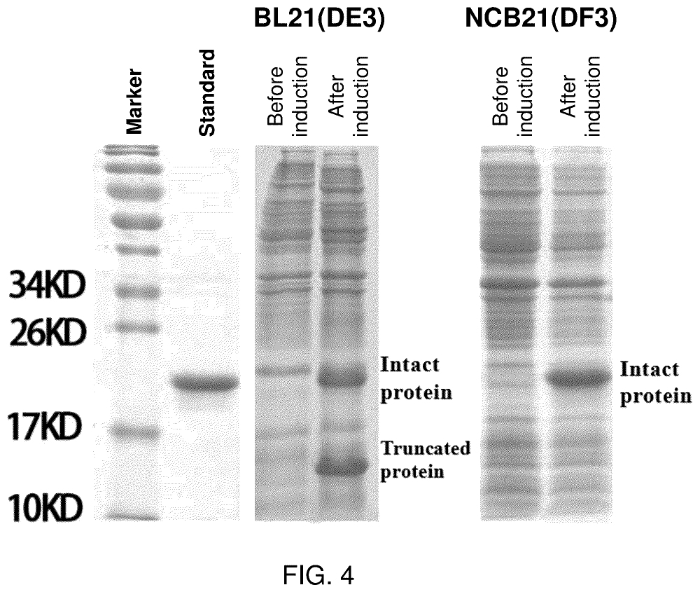

NCB21 (DE3) competent cells were transformed with plasmid pET21a-rhGH-K145. The plasmid pET21a-rhGH-K145 was constructed by inserting the coding reading frame of recombinant human growth hormone rhGH at the multiple cloning site of commercially available pET21a plasmid (Novagen), and the codon of the 145th amino acid of rhGH was replaced with amber codon UAG. Transformed single clones were selected and inoculated into an LB culture medium containing ampicillin 100 mg/L, spectinomycin 100 mg/L, kanamycin 100 mg/L, and arabinose 0.5 g/L, and cultured overnight at 180 rpm at 30° C. The activated bacterial solution was inoculated into a fresh LB culture medium containing ampicillin 100 mg/L, spectinomycin 100 mg/L, kanamycin 100 mg/L and arabinose 0.1 g/L at an inoculation amount of 1/100, and incubated with shaking at 37° C., 180 r/min until the OD600 was about 0.5-0.8. Inducer IPTG was added at a final concentration of 1 mM, an unnatural amino acid NAEK was added at a final concentration of 1 mM, and the induction culture was performed at 37° C., 180 r/min for 24 h. After induction, 1 mL of the bacterial solution was centrifuged at 12000 g. The supernatant was discarded and the bacteria were collected, resuspended in 100 μL of PBS, and verified by SDS-PAGE gel electrophoresis. The results are shown in FIG. 4. When using NCB21 (DE3) for expression, essentially no truncated proteins were produced, whereas the BL21 (DE3) strain, as a control, produced a large amount of truncated proteins when co-transformed with helper plasmid pUltra-py1RS and expression plasmid pET21a-rhGH-K145.

The above are only preferred embodiments of the present invention. It should be noted that those of ordinary skill in the art can also make several improvements and modifications without departing from the principle of the present invention, and such improvements and modifications shall fall within the protection scope of the present invention.

1. A method for constructing a strain for producing a recombinant protein containing unnatural amino acids, comprising: modifying the expression of a prfA gene contained in the strain to be controllable.

2. The method according to claim 1, wherein the expression of the prfA gene in the genome of the strain is modified to be suppressed, and a plasmid containing the prfA gene and capable of expressing RF1 is introduced into the strain, wherein the expression or replication of the prfA gene in the plasmid is controlled.

3. The method according to claim 2, wherein the promoter of the prfA gene in the plasmid is an inducible expression promoter; preferably, the inducible expression promoter is a tac promoter, a lacUV5 promoter, a T7 promoter, a T5 promoter, an araBAD promoter, or a pR/pL promoter; and/or
the replication origin of the prfA gene in the plasmid is a conditional deletion-type replication origin; preferably, the conditional deletion-type replication origin is a high-temperature conditional deletion-type replication origin pSG5 or pSC101.

4. The method according to claim 2 or 3, wherein the modification of the expression of the prfA gene in the genome to be suppressed is achieved by gene knockout, gene silencing, or gene mutation.

5. The method according to claim 1, wherein the expression of the prfA gene in the genome of the strain is modified to be inducible, and the promoter of the prfA gene in the genome of the strain is replaced by an inducible expression promoter; preferably, the inducible expression promoter is a tac promoter, a lacUV5 promoter, a T7 promoter, a T5 promoter, an araBAD promoter, or a pR/pL promoter.

6. The method according to any one of claims 1-5, wherein the method further comprises: introducing a plasmid encoding an unnatural amino acid tRNA/tRNA synthetase orthogonal to a natural amino acid tRNA/tRNA synthetase into the strain; preferably, the plasmid encoding an unnatural amino acid tRNA/tRNA synthetase orthogonal to a natural amino acid tRNA/tRNA synthetase is a pUltra-py1RS helper plasmid.

7. The method according to claim 6, wherein the strain is Escherichia coli BL21 (DE3), and the method comprises:
(1) knocking out the prfA gene from the genome of the strain by CRISPR technology;
(2) constructing a plasmid containing a pSG5 replication origin, an araBAD promoter, and a prfA gene; and
(3) transferring the plasmid in step (2) and the pUltra-py1RS helper plasmid into the strain obtained in step (1).

8. A strain for producing a recombinant protein containing unnatural amino acids, wherein the strain is capable of expressing an unnatural amino acid tRNA/tRNA synthetase orthogonal to a natural amino acid tRNA/tRNA synthetase, and the expression of a prfA gene in the strain is controlled; preferably, the strain is constructed by the method according to any one of claims 1-7.

9. A method for producing a recombinant protein containing unnatural amino acids, comprising: introducing a gene encoding the recombinant protein into the strain according to claim 8, and culturing the strain to express the recombinant protein.

10. The method according to claim 9, wherein the method comprises: 1) constructing a strain with controlled prfA gene expression; 2) introducing a helper plasmid expressing an unnatural amino acid tRNA/tRNA synthetase and an expression plasmid expressing the target recombinant protein into the strain; 3) inducing the expression of the prfA gene during the strain growth stage; and inducing the prfA gene to stop expression and inducing the recombinant protein to express in the recombinant protein expression stage.
